(19) United States
(12) Patent Application Publication
(10) Pub. No.: US 2008/0177371 A1
(43) Pub. Date: Jul. 24, 2008

(54) IMPLANTABLE DEVICES AND METHODS OF FORMING THE SAME

(75) Inventors: S. Eric Ryan, Hopkinton, MA (US); Richard Sahagian, Burlington, MA (US)

Correspondence Address: MILLS & ONELLO LLP, ELEVEN BEACON STREET, SUITE 605, BOSTON, MA, 02108, US

(73) Assignee: CorNova, Inc., Burlington, MA (US)

(21) Appl. No.: 11/843,376

(22) Filed: Aug. 22, 2007

Related U.S. Application Data
(60) Provisional application No. 60/823,692, filed on Aug. 28, 2006.
(60) Provisional application No. 60/825,434, filed on Sep. 13, 2006.
(60) Provisional application No. 60/895,924, filed on Mar. 20, 2007.
(60) Provisional application No. 60/941,813, filed on Jun. 4, 2007.

(51) Int. Cl. A61F 2/06; A61L 33/02; C23C 14/28

(52) U.S. Cl. 623/1.15; 623/1.46; 427/2.25; 427/595

(57) ABSTRACT

An implantable device and method of forming the same comprises a substrate, an adhesion layer, and a capping layer. The adhesion layer comprises a portion with a predominant proportion of palladium, the portion of the predominant proportion of palladium directly on the substrate. The capping layer comprises a capping layer material, and is on the adhesion layer.

### RELATED APPLICATIONS

This application claims the benefit of U.S. Patent Application No. 60/823,692 filed on 28 Aug. 2006, entitled “Adhesive Surfaces for Implanted Devices,” U.S. Patent Application No. 60/825,434 filed on 13 Sep. 2006, entitled “Flexible Expandable Stent,” U.S. patent application Ser. No. 11/613,443 filed on 20 Dec. 2006, entitled “Flexible Expandable Stent,” U.S. Patent Application No. 60/895,924 filed on 20 Mar. 2007, entitled “Implantable Devices and Methods of Forming the Same,” and U.S. Patent Application No. 60/941,813 filed on Jun. 4, 2007 entitled “Implantable Devices Having Textured Surfaces and Method of Forming the Same,” the contents of each being incorporated herein in their entirety by reference.

This application is related to U.S. Ser. No. ______, filed on or around the filing date of the present application, entitled “Implantable Devices Having Textured Surfaces and Methods of Forming the Same,” by Richard Sahagian and S. Eric Ryan, the contents incorporated herein in their entirety by reference.

### FIELD OF THE INVENTION

The present invention relates to implantable devices and, in particular, to implantable devices including adhesive layers that adhere a biocompatible capping layer to a device substrate, and methods of forming the same.

### BACKGROUND OF THE INVENTION

Implantable devices provide for the treatment of a myriad of conditions and include devices for heart control and support, muscular-skeletal support, and intravascular support. The surfaces of these devices generally require a significant level of biocompatibility, including stability, smoothness, and resistance to undesired biological interaction. Stents, for example, are implantable prostheses used to maintain and reinforce vascular and endoluminal ducts in order to treat and prevent a variety of cardiovascular conditions. Typical uses include maintaining and supporting coronary arteries after they are opened and unblocked, such as through an angioplasty operation.

As a foreign object inserted into a vessel, a stent can potentially impede the flow of blood. This effect can be exacerbated by the undesired growth of tissue on and around the stent, potentially leading to complications including thrombosis and restenosis. Typical stents have the basic form of an open-ended tubular element supported by a mesh of thin struts with openings formed between the struts. Designs typically include strong, flexible, and ductile base substrate materials. Some stents also include metallic outer layers such as gold or platinum in order to either increase the radiopacity of the stent and/or improve its biocompatibility in order to promote proper healing of tissue about the stent upon its deployment. In order to further resist excessive tissue growth, some stents include active drug-eluting polymer coatings. However, as further described below, traditional techniques of applying these layers to certain substrates fail to adhere them sufficiently to the device, thus creating safety risks which could outweigh the potential benefits. Most stents are manufactured to be reliably deformable in crimped and deployed states. Prior to deployment, a stent is generally in a crimped state and secured about an expandable balloon at the distal end of a catheter. When inserted into position, the balloon and stent are expanded, thus deforming the stent struts and bending the stent along the inner walls of the vessel. The crimping and expansion process may thus subject any coating materials to additional stresses, increasing the likelihood that the coating undergoes flaking and cracking.

Various biocompatible metallic materials, for example, platinum or gold, can be applied onto conventional stents using various techniques including the use of metal bands, electrochemical deposition, and ion beam assisted deposition. However, metal bands are prone to becoming loose, shifting, or otherwise separating from the stent. Moreover, a metal band around a stent can cause abrasions to the intima (i.e., the lining of a vessel wall) during insertion of the device, especially if the bands have sharp edges or outward projections. The physiological response can often be a reclosure of the lumen, thereby negating the beneficial effects of the device. Additionally, cellular debris can be trapped between the intravascular device and the band, and the edges of the band can serve as a site for thrombosis formation.

Electrochemical deposition, including chemical vapor deposition (CVD), physical vapor deposition (PVD), or electroplating, may result in fairly porous stent surface layers, with densities on the order of about 70-75% of full bulk density, or may not provide sufficient adhesion for purposes of medical device applications.

Ion beam assisted deposition (IBAD) of radiopaque materials can be used to improve the adhesion of coatings to the substrate surface. IBAD employs conventional PVD to create a vapor of atoms of, for instance, a noble metal that coats the surface of the substrate, while simultaneously bombarding the substrate surface with ions at energies, typically in the range of 0.8 to 1.5 keV, to impact and condense the metal atoms on the substrate surface. An independent ion source is used as the source of ions.

Coatings produced by IBAD techniques, however, are costly. When evaporating, atoms of expensive noble metals are emitted over a large solid angle compared to that subtended by the device or devices being coated, thus requiring a costly reclaiming process. Moreover, because an evaporator uses a molten metal, it must be located upright on the floor of the deposition chamber to avoid spilling, thereby restricting the size and configuration of the chamber and the devices being coated. Additionally, evaporators cannot deposit mixtures of alloys effectively because of the differences in the alloy components' evaporation rates. As such, the composition of the resulting coating constantly changes.

Furthermore, the conventional IBAD approach is applied by directing the flux of bombarding ions from a location significantly separated from the evaporant, i.e., atoms of metal being deposited, in a non-linear manner, that is, the bombarding ions and metal atoms approach the substrate from different directions. To this end, the energy from the bombarding ions transferred to the evaporant atoms varies depending on the extent to which the two streams overlap. In addition, the number of bombarding ions can be relatively few in number although high in energy, resulting in the metal atoms likely being either implanted tightly into their original impact point or back-sputtering off of the substrate surface. As a result, the growth mechanism of the coating can be inconsistent, and uniform coating properties are difficult to achieve. Moreover, these methods are generally only able to achieve densities of between about 92% to less than 95% of full bulk density.

Techniques have also been developed for providing radiopaque surfaces on stents, which enhance the detectability or visualization of what may have been otherwise undetectable core strut materials, and are principally directed toward providing surfaces viewable by fluoroscopes, which requires relatively substantial quantities of radiopaque material, for example, gold, over the substrate surface of the stent, thereby requiring the surfaces to have increased surface dimensions, such as an increased surface area and an increased radiopaque layer thickness generally requiring a thickness greater than 25,000 angstroms. Here, the resulting stent has a larger surface area and is more susceptible to thrombosis or other adverse medical conditions. Although certain core materials (e.g., cobalt-chromium and steel alloys) can provide sufficient radiopacity without the need for additional radiopaque layers, these materials may lack preferable biocompatibility. Furthermore, the above-described techniques and/or combinations of materials for coating stents can only provide suboptimal degrees of purity, adhesion, thinness, and/or uniformity of preferred biocompatible capping materials (e.g. titanium, silver, nickel, gold, and platinum) to typical substrate materials. Other technologies have adopted the discussed methods to provide textured metallic surfaces for directly bonding with polymers, therapeutic agents and/or other materials. These technologies are similarly constrained by non-adherent, relatively thick and/or uneven layers with less than optimal biocompatibility over a substrate surface.

### SUMMARY OF THE INVENTION

Embodiments of the present invention are directed to implantable devices and methods of manufacturing the same, which overcome the limitations associated with the aforementioned approaches. In particular, embodiments provide improved combinations of substrate materials, including highly radiopaque materials, with adherent, thin, uniform, and biocompatible coatings and methods for their manufacture.

In accordance with one aspect, an implantable device comprises a substrate, an adhesion layer, and a capping layer. The adhesion layer comprises a portion with a predominant proportion of palladium, in which the portion of the adhesion layer with a predominant proportion of palladium is directly on the substrate. The capping layer comprises a capping layer material and is on the adhesion layer.

In an embodiment, the capping layer material comprises a biocompatible material. In another embodiment, the biocompatible material comprises at least one of platinum, platinum-iridium, tantalum, titanium, and alloys thereof. In an embodiment, the biocompatible material comprises at least one of tin, indium, palladium, gold, and alloys thereof.

In another embodiment, the capping layer material comprises a predominant proportion of platinum.

In another embodiment, the adhesion layer between the substrate and the capping layer has a thickness of less than about 5000 angstroms.

In another embodiment, at least one of the capping layer and the adhesion layer has a thickness between about 100 and 5000 angstroms.

In another embodiment, the capping layer has a thickness of less than about 2500 angstroms.

In another embodiment, at least one of the capping layer and the adhesion layer has a thickness between about 500 and 2500 angstroms.

In another embodiment, a transition between the adhesion layer and the substrate has a thickness of about 10 atomic thicknesses or less.

In another embodiment, a transition between the adhesion layer and the substrate has a thickness of about 5 atomic thicknesses or less.

In another embodiment, at least one of the adhesion layer and the capping layer is substantially of a density greater than about 95% full bulk density.

In another embodiment, at least one of the adhesion layer and the capping layer is substantially of a density equal to or greater than about 97% full bulk density.

In another embodiment, the substrate comprises a highly radiopaque material. In another embodiment, the highly radiopaque material comprises cobalt-chromium material. In another embodiment, the substrate comprises a metallic material including at least one of stainless steel, nickel-based steel, cobalt-chromium, titanium, nitinol, and alloys thereof.

In another embodiment, the adhesion layer comprises a first portion that is directly on the substrate and a second portion that is directly on the first portion, and wherein the second portion is between the first portion and the capping layer. In another embodiment, the second portion comprises a gradated mixture of palladium and capping layer material, wherein the gradated mixture of palladium and capping layer material includes a high concentration of palladium and a low concentration of capping layer material in a region proximal to the first portion of the adhesion layer, and wherein the gradated mixture of palladium and capping layer material includes a low concentration of palladium and a high concentration of capping layer material in a region proximal to the capping layer.

In another embodiment, the capping layer is directly on the adhesion layer.

In another embodiment, the adhesion layer comprises a predominant proportion of palladium throughout its thickness.

In another embodiment, a transition between the capping layer and the adhesion layer has a thickness of about 10 atomic thicknesses or less.

In another embodiment, a transition between the capping layer and the adhesion layer has a thickness of about 5 atomic thicknesses or less.

In another embodiment, the capping layer material comprises a material other than palladium.

In another embodiment, the device further comprises a polymer layer on the capping layer.

In another embodiment, the device comprises a flexible body.

In another embodiment, the device comprises an intravascular stent.

In another embodiment, the body of the intravascular stent is a flexible expandable body of interconnected struts.

In accordance with another aspect, an implantable device comprises a substrate, an adhesion layer, and a capping layer. The adhesion layer comprises a portion with a predominant proportion of gold, and the portion of the adhesion layer with a predominant proportion of gold is directly on the substrate. The capping layer comprises a capping layer material, and the capping layer on the adhesion layer. The adhesion layer between the substrate and the capping layer has a thickness of less than about 5000 angstroms.

In an embodiment, the capping layer material comprises a biocompatible material. In another embodiment, the biocompatible material comprises at least one of platinum, platinum-iridium, tantalum, titanium, and alloys thereof. In an embodiment, the biocompatible material comprises at least one of tin, indium, palladium, gold, and alloys thereof.

In another embodiment, the capping layer material comprises a predominant proportion of platinum.

In another embodiment, at least one of the capping layer and the adhesion layer has a thickness between about 100 and 5000 angstroms.

In another embodiment, the capping layer has a thickness of less than about 2500 angstroms.

In another embodiment, at least one of the capping layer and the adhesion layer has a thickness between about 500 and 2500 angstroms.

In another embodiment, a transition between the adhesion layer and the substrate has a thickness of about 10 atomic thicknesses or less.

In another embodiment, a transition between the adhesion layer and the substrate has a thickness of about 5 atomic thicknesses or less

In another embodiment, at least one of the adhesion layer and the capping layer is substantially of a density greater than about 95% full bulk density.

In another embodiment, at least one of the adhesion layer and the capping layer is substantially of a density equal to or greater than about 97% full bulk density.

In another embodiment, the substrate is radiopaque. In another embodiment, the substrate comprises a highly radiopaque material. In another embodiment the highly radiopaque material includes cobalt-chromium material.

In another embodiment, the substrate comprises a metallic material including at least one of stainless steel, nickel-based steel, cobalt-chromium, titanium alloys, nitinol, and alloys thereof.

In another embodiment, the adhesion layer comprises a first portion that is directly on the substrate and a second portion that is directly on the first portion, and the second portion is between the first portion and the capping layer.

In another embodiment, the second portion comprises a gradated mixture of gold and capping layer material, wherein the gradated mixture of gold and capping layer material includes a high concentration of gold and a low concentration of capping layer material in a region proximal to the first portion of the adhesion layer, and the gradated mixture of gold and capping layer material includes a low concentration of gold and a high concentration of capping layer material in a region proximal to the capping layer.

In another embodiment, the capping layer is directly on the adhesion layer.

In another embodiment, the adhesion layer comprises a predominant proportion of gold throughout its thickness.

In another embodiment, a transition between the capping layer and the adhesion layer has a thickness of about 10 atomic thicknesses or less.

In another embodiment, a transition between the capping layer and the adhesion layer has a thickness of about 5 atomic thicknesses or less.

In another embodiment, the adhesion layer comprises a material other than gold.

In another embodiment, the device further comprises a polymer layer on the capping layer.

In another embodiment, the implantable device comprises a flexible body.

In another embodiment, the implantable device is an intravascular stent.

In another embodiment, the body of the intravascular stent is a flexible expandable body of interconnected struts.

In accordance with another aspect, a method of providing a surface on an implantable device comprises providing a substrate of the implantable device, providing an adhesion layer comprising a portion with a predominant proportion of palladium directly on the substrate by simultaneously directing a flux of palladium atoms and a flux of bombarding ions toward the substrate, and providing a capping layer comprising a capping layer material on the adhesion layer by directing a flux of capping layer material atoms and a flux of bombarding ions toward the provided adhesion layer.

In an embodiment, the bombarding ions are directed in substantially collinear fashion toward the substrate with respect to the fluxes of palladium or capping material atoms.

In an embodiment, providing the adhesion layer comprises providing a first portion of the adhesion layer directly on the substrate, the first portion of the adhesion layer comprising the predominant proportion of palladium, and providing a second portion of the adhesion layer directly on the first portion, the second portion comprising a gradated mixture of palladium and capping layer material between the first portion and the capping layer.

In another embodiment, the gradated mixture includes a high concentration of palladium and a low concentration of capping layer material in a region proximal to the first portion of the adhesion layer by providing a greater proportion of palladium atoms than capping layer material atoms, and wherein the gradated mixture includes a low concentration of palladium and a high concentration of capping layer material in a region proximal to the capping layer by providing a greater proportion of capping layer material atoms than palladium atoms.

In another embodiment, the gradated mixture is provided by simultaneously directing a flux of palladium atoms, a flux of capping layer material atoms, and fluxes of bombarding ions toward the substrate.

In another embodiment, forming the adhesion layer comprises using at least one magnetron to direct fluxes of palladium atoms and the capping layer material atoms. In another embodiment, the at least one magnetron comprises an unbalanced magnetron.

In another embodiment, the capping layer is substantially biocompatible.

In another embodiment, the capping layer material atoms are platinum atoms.

In another embodiment, the adhesion layer between the substrate and the capping layer has a thickness of less than about 5000 angstroms.

In another embodiment, at least one of the capping layer and the adhesion layer has a thickness between about 100 and 5000 angstroms.

In another embodiment, at least one of the capping layer and the adhesion layer has a thickness of less than about 2500 angstroms.

In another embodiment, a transition between the substrate and the adhesion layer has a thickness of about 10 atomic thicknesses or less.

In another embodiment, a transition between the substrate and the adhesion layer has a thickness of about 5 atomic thicknesses or less.

In another embodiment, providing the capping layer comprises forming the capping layer directly on the adhesion layer.

In another embodiment, providing the adhesion layer comprises providing the adhesion layer to comprise a predominant proportion of palladium throughout its thickness.

In another embodiment, a transition between the adhesion layer and the capping layer has a thickness of about 10 atomic thicknesses or less.

In another embodiment, a transition between the adhesion layer and the capping layer has a thickness of about 5 atomic thicknesses or less.

In another embodiment, the adhesion layer is substantially of a density greater than about 95% full bulk density.

In another embodiment, the capping layer is substantially of a density greater than about 95% full bulk density.

In another embodiment, the adhesion layer is of a density equal to or greater than about 97% full bulk density.

In another embodiment, the capping layer is of a density equal to or greater than about 97% full bulk density.

In accordance with another aspect, a method of providing a surface on an implantable device comprises providing a substrate of the implantable device, providing an adhesion layer comprising a portion with a predominant proportion of gold directly on the substrate by simultaneously directing a flux of gold atoms and a flux of bombarding ions toward the substrate, and providing a capping layer comprising a capping layer material on the adhesion layer by directing a flux of capping layer material atoms and a flux of bombarding ions toward the provided adhesion layer, the adhesion layer between the substrate and the capping layer having a thickness of less than about 5000 angstroms.

In an embodiment, the bombarding ions are directed in substantially collinear fashion toward the substrate with respect to the fluxes of gold or capping material atoms.

In an embodiment, providing the adhesion layer comprises providing a first portion of the adhesion layer directly on the substrate, the first portion of the adhesion layer comprising the predominant proportion of gold, and providing a second portion of the adhesion layer directly on the first portion, the second portion comprising a gradated mixture of gold and capping layer material between the first portion and the capping layer.

In another embodiment, the gradated mixture includes a high concentration of gold and a low concentration of capping layer material in a region proximal to the first portion of the adhesion layer by providing a greater proportion of gold atoms than capping layer material atoms, and the gradated mixture includes a low concentration of gold and a high concentration of capping layer material in a region proximal to the capping layer by providing a greater proportion of capping layer material atoms than the gold atoms.

In another embodiment, the gradated mixture is provided by simultaneously directing a flux of gold atoms, a flux of capping layer material atoms, and fluxes of bombarding ions toward the substrate.

In another embodiment, forming the adhesion layer comprises using at least one magnetron to control proportions of the gold atoms and the capping layer material atoms. In another embodiment, the at least one magnetron comprises an unbalanced magnetron.

In another embodiment, the capping layer is substantially biocompatible.

In another embodiment, the capping layer material atoms are platinum atoms.

In another embodiment, at least one of the capping layer and the adhesion layer has a thickness between about 100 and 5000 angstroms.

In another embodiment, at least one of the capping layer and the adhesion layer has a thickness of less than about 2500 angstroms.

In another embodiment, a transition between the substrate and the adhesion layer has a thickness of about 10 atomic thicknesses or less.

In another embodiment, a transition between the substrate and the adhesion layer has a thickness of about 5 atomic thicknesses or less.

In another embodiment, providing the capping layer comprises forming the capping layer directly on the adhesion layer.

In another embodiment, providing the adhesion layer comprises providing the adhesion layer to comprise a predominant proportion of gold throughout its thickness.

In another embodiment, a transition between the adhesion layer and the capping layer has a thickness of about 10 atomic thicknesses or less.

In another embodiment, a transition between the adhesion layer and the capping layer has a thickness of about 5 atomic thicknesses or less.

In another embodiment, the adhesion layer is of a density greater than about 95% full bulk density.

In another embodiment, the capping layer is of a density greater than about 95% full bulk density.

In another embodiment, the adhesion layer is of a density equal to or greater than about 97% full bulk density.

In another embodiment, the capping layer is of a density equal to or greater than about 97% full bulk density.

In another embodiment, at least one of the capping layer or adhesion layer is of a density equal to or greater than about 97% full bulk density.

In accordance with another aspect, an implantable device comprises a substrate comprising cobalt-chromium and a biocompatible coating having a thickness of less than about 15,000 angstroms that is directly on the substrate.

In an embodiment, the present invention is directed to the biocompatible coating comprises at least one of a capping layer and an adhesion layer.

In another embodiment, the capping layer comprises at least one of platinum, platinum-iridium, and alloys thereof.

In another embodiment, the capping layer comprises a predominant proportion of platinum.

In another embodiment, the biocompatable coating has a thickness of less than about 10,000 angstroms.

In another embodiment, the biocompatable coating has a thickness of between about 2,500 and 5,000 angstroms.

In another embodiment, the biocompatable coating has a thickness of less than about 2500 angstroms.

In another embodiment, the biocompatable coating has a thickness of less than about 500 angstroms.

In another embodiment, the biocompatible coating is of a density greater than about 95% full bulk density.

In another embodiment, the biocompatible coating is of a density greater than or equal to about 97% full bulk density.

In accordance with another aspect, an implantable device comprises a substrate, an adhesion layer comprising a predominant proportion of palladium, wherein a transition between the substrate and the adhesion layer has a thickness of about 10 atomic thicknesses or less, and a capping layer comprising a capping layer material, the capping layer on the adhesion layer.

In accordance with another aspect, an implantable device comprises a substrate, an adhesion layer comprising a predominant proportion of gold, wherein a transition between the substrate and the adhesion layer has a thickness of about 10 atomic thicknesses or less, and a capping layer comprising a capping layer material, the capping layer on the adhesion layer, wherein the adhesion layer between the substrate and the capping layer has a thickness of less than about 5000 angstroms.

In accordance with another aspect, a method of forming a surface on an implantable device comprises providing a substrate of the implantable device, providing an adhesion layer having a thickness of less than about 5000 angstroms that comprises a predominant proportion of palladium on the substrate by simultaneously directing a flux of palladium atoms and a flux of bombarding ions toward the substrate, and

providing a capping layer comprising a capping layer material on the adhesion layer by directing a flux of capping layer material atoms and a flux of bombarding ions toward the provided adhesion layer.

In accordance with another aspect, a method of forming a surface on an implantable device comprises providing a substrate of the implantable device, providing an adhesion layer having a thickness of less than about 5000 angstroms that comprises a predominant proportion of gold on the substrate by simultaneously directing a flux of gold atoms and a flux of bombarding ions toward the substrate, wherein a transition between the substrate and the adhesion layer has a thickness of about 10 atomic thicknesses or less, and providing a capping layer comprising a capping layer material on the adhesion layer by directing a flux of capping layer material atoms and a flux of bombarding ions toward the provided adhesion layer.

### BRIEF DESCRIPTION OF THE DRAWINGS

The structure, operation, and methodology of the embodiments of the invention, together with other objects and advantages thereof, may best be understood by reading the following detailed description in connection with the drawings in which each part has an assigned numeral or label that identifies it wherever it appears in the various drawings. The drawings are not necessarily to scale, emphasis instead being placed upon illustrating the principles of the invention.

FIG. 1 is an illustrative cross-sectional view of a layered surface of an implantable device in accordance with an embodiment of the invention.

FIG. 2A is an illustrative side view of a stent in accordance with an embodiment of the invention. FIG. 2B is an illustrative transverse cross-sectional view of a strut of the stent of FIG. 2A, taken along section lines I-I′ of FIG. 2A.

FIG. 3 is an illustrative cross-sectional view of a surface of an implantable device in accordance with an embodiment of the invention.

FIG. 4 is an illustrative cross-sectional view of a surface of an implantable device in accordance with another embodiment of the invention.

FIG. 5 is an illustrative view of surface layers being formed on a substrate of an implantable device in accordance with an embodiment of the invention.

FIG. 6 is a side-perspective illustrative schematic of an apparatus for coating an implantable device using multiple magnetrons according to an embodiment of the invention.

### DETAILED DESCRIPTION OF EMBODIMENTS OF THE INVENTION

The accompanying drawings are described below, in which example embodiments in accordance with the present invention are shown. Specific structural and functional details disclosed herein are merely representative. The invention may be embodied in many alternative forms and should not be construed as limited to the example embodiments described herein.

It will be understood that the drawings are not intended to accurately reflect relative proportions of layer thicknesses but rather to illustrate the general order of layer positions.

Accordingly, specific embodiments are shown by way of example in the drawings. It should be understood, however, that there is no intent to limit the invention to the particular forms disclosed herein, but to the contrary, the invention is to cover all modifications, equivalents, and alternatives falling within the spirit and scope of the claims.

It will be understood that, although the terms first, second, etc. may be used herein to describe various elements, these elements should not be limited by these terms. These terms are used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element is referred to as being “on,” “adjacent,” “connected to,” or “coupled to” another element, it can be directly on, connected to or coupled to the other element or intervening elements may be present. In contrast, when an element is referred to as being “directly on,” “directly adjacent,” “directly connected to,” or “directly coupled to” another element, there are no intervening elements present. Other words used to describe the relationship between elements should be interpreted in a like fashion (e.g., “between” versus “directly between,” etc.).

It will be understood that the term “directly on,” as used herein, is intended to describe situations where there is a substantial molecular contact between two elements or layers, for example, between an adhesion layer and a substrate, or between a capping layer and a substrate.

It will be understood that the term “gradated mixture,” as used herein, refers to a layer having a composition gradiant comprising a mixture of at least first and second materials, wherein there is a smooth, continuous composition gradient from one side of the layer to the other side such that the ratio of first material to second material is relatively higher at one side and lower at the other side.

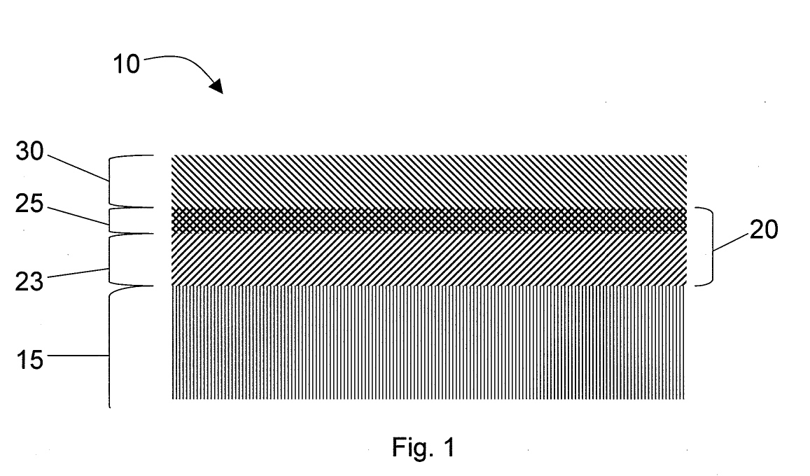

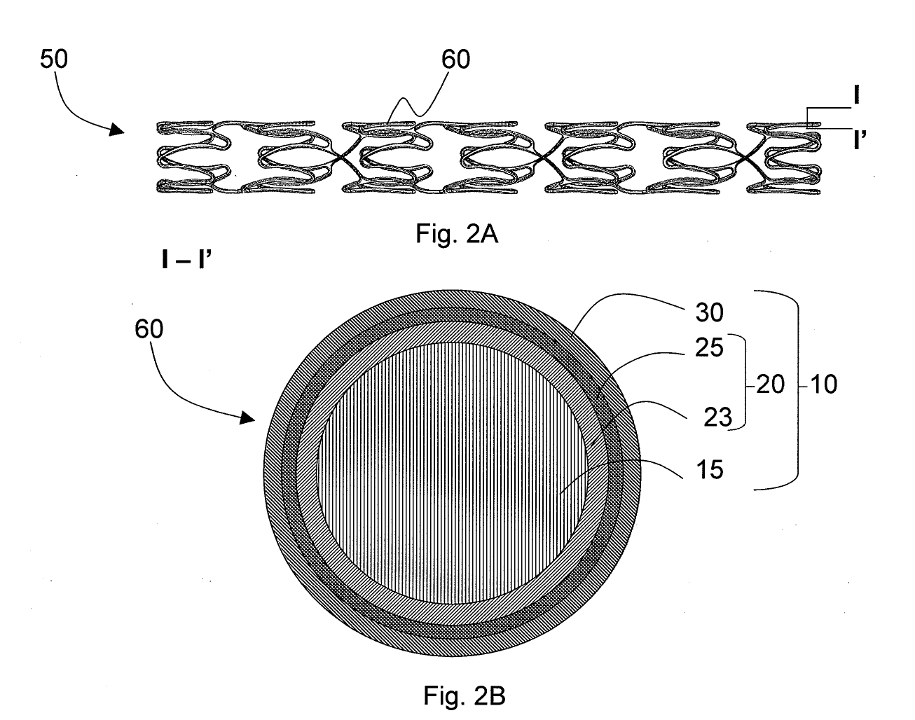

FIG. 1 is an illustrative cross-sectional view of a layered surface 10 of an implantable device in accordance with an embodiment of the invention. FIG. 2A is an illustrative side view of a stent 50 including such layered outer surfaces in accordance with an embodiment of the invention. FIG. 2B is an illustrative transverse cross-sectional view of a strut 60 of the stent 50 of FIG. 2A, taken along section lines I-I′ of FIG. 2A.

As shown in the embodiments of FIGS. 1, 2A, and 2B, a body of an implantable device includes a substrate 15. An adhesion layer 20 is provided on the substrate 15, and a capping layer 30 is provided on the adhesion layer 20. Examples of the manner in which the capping layer 30 and adhesion layer 20 can be applied are described in detail below.

The substrate 15 can be formed of any number of applicable materials known to one of ordinary skill, for example, stainless steel, nickel-based steel, cobalt-chromium, titanium, nitinol, and alloys thereof. In an embodiment, the substrate 15 includes materials that provide properties permitting the implantable device to be detected by radiography or fluoroscopy when the device is positioned inside the human body, for example, highly radiopaque materials known to one of skill in the art. A highly radiopaque material can generally provide a core structure in a low-profile device such as a stent without the need for additional radiopaque coatings.

In an embodiment, a substrate comprising a predominant proportion of cobalt-chromium material is well-suited for this purpose. Cobalt-chromium material can include pure cobalt-chromium or various cobalt-chromium alloys such as, for example, L605 (Co-20Cr-15W-10Ni), MP35N (35Co-35Ni-20Cr-10Mo), Phynox (40Co-20Cr-16Fe-15Ni-7Mo—), and Elgiloy (40Co-20Cr-16Fe-15Ni-7Mo—). The substrate materials need not be particularly biocompatible, but are preferred to be designed for particular beneficial features, including material strength, flexibility, radiopacity, and malleability, depending on the application. For instance, in the case of the stent 50 shown in FIGS. 2A-2B, the materials used to provide a stent body must be sufficiently strong, expandable, and permit the retaining of sufficient radial forces after deployment.

In the embodiments illustrated at FIGS. 1, 2A, and 2B, the adhesion layer 20 includes at least one of a first portion 23 and an optional second portion 25. The first portion 23 of the adhesion layer 20 is directly on the substrate 15. In an embodiment, the first portion 23 of the adhesion layer 20 consists essentially of adhesion layer materials to permit a strong bond to the substrate surface 15, such as palladium or gold, for example, 100% palladium or gold, or nearly 100% palladium or gold, or a mixture of palladium and gold, and comprises little or no capping layer material. In another embodiment, the first portion 23 of the adhesion layer 20 comprises a predominant proportion of adhesion layer material, for example, at least 50% palladium or gold. Palladium, in particular, can provide a very strong bond between a substrate such as cobalt-chromium material and a capping material. As well as providing a strong bond between a substrate and a capping layer, an adhesion layer, particularly one including palladium material, can act as a strong diffusion barrier between a substrate and the exterior of the device, thus helping prevent the escape of potentially toxic and less biocompatible materials such as, for example, cobalt-chromium material and its components and reactive by-products (e.g. resulting from metal ion diffusion).

In an embodiment, a transition between the adhesion layer 20 and the substrate 15 has a thickness of about 10 atomic thicknesses or less. In another embodiment, the transition between the adhesion layer 20 and the substrate 15 has a thickness of about 5 atomic thicknesses or less. Preferably, the transition between the adhesion layer 20 and the substrate 15 has a thickness of about 2 atomic thicknesses or less.

In the embodiment shown in FIG. 1, the second portion 25 of the adhesion layer 20 is between the capping layer 30 and the first portion 23. In an embodiment, a region of the second portion 25 adjacent the capping layer 30 comprises a predominant proportion of capping layer material such as platinum, for example, nearly 100% platinum, or at least 50% platinum, which permits a strong bond to the capping layer 30. In another embodiment, the region of the second portion 25 adjacent the capping layer 30 consists essentially of capping layer material, for example, platinum and/or alloys thereof. Additional capping layer materials can include, for example, platinum-iridium, tantalum, titanium, tin, indium, palladium, gold and alloys thereof, many of which provide strong biocompatibility. Some examples of alloys containing the aforementioned materials include, for example, TiAl6V4, TiAl5Fe2.5, Pd79Au10, Au75Pd19, Au61Pd29.

In another embodiment, the second portion 25 of the adhesion layer 20 comprises a gradated mixture of adhesion layer material, such as palladium or gold, and capping layer material, such as what is present in the capping layer 30. Specifically, the second portion 25 of the adhesion layer 20 transitions from a high concentration of adhesion layer material and a low concentration of capping layer material at a region adjacent the first portion 23 of the adhesion layer 20 to a low concentration of adhesion layer material and a high concentration of capping layer material at a region adjacent the capping layer 30.

In an embodiment, the layered surface 10 includes a substrate 15 which is radiopaque that comprises a predominant proportion of a highly radiopaque material such as, for example, cobalt-chromium material, a first portion 23 of an adhesion layer 20 comprising a predominant proportion of palladium, and a capping layer 30 comprising a predominant proportion of platinum. The second portion 25 of the adhesion layer 20 between the first portion 23 and the capping layer 30 comprises a gradated mixture of palladium and platinum.

In another embodiment, the layered surface 10 includes a substrate 15 comprising a predominant proportion of a radiopaque material, for example, cobalt-chromium material, a first portion 23 of an adhesion layer 20 comprising a predominant proportion of gold, and a capping layer 30 comprising a predominant proportion of platinum. A second portion 25 of the adhesion layer 20 between the first portion 23 and the capping layer 30 comprises a gradated mixture of gold and platinum.

In an embodiment, the thickness of the substrate can be about 80 or more microns thick, wherein enough of the highly radiopaque material (e.g. cobalt-chromium material) is present to make the substrate radiopaque while providing other desired bio-mechanical properties (e.g. flexibility, strength, etc . . . ) for a stent device. The selected layer thickness depends in part on the content and shape of the substrate surface. For instance, designs having sharper and more angular features may require greater layer thicknesses for proper adhesion and protection. In an embodiment, the adhesion layer 20 has a thickness of less than 5000 angstroms. In another embodiment, the adhesion layer 20 has a thickness in the range of approximately 100 to 5000 angstroms, and preferably less than about 2500 angstroms, or otherwise sufficient to provide adequate bonding between the capping layer 30 and the substrate 15 while preserving the flexibility and formability of the stent. In another embodiment, the adhesion layer 20 has a thickness between about 500 and 2500 angstroms. In the embodiments illustrated above, the second portion 25 of the adhesion layer 20 has a thickness in the range of a few atoms in thickness to about 2000 angstroms.

In an embodiment, the capping layer 30 has a thickness in the range of approximately 100 to 5000 angstroms. In another embodiment, the capping layer 30 can have a thickness that is less than 2500 angstroms, or otherwise sufficient to provide an adequate barrier between tissue material and the adhesion layer 20 and/or substrate 15.

A stent or other medical device fabricated in accordance with the embodiments described herein can have a highly radiopaque substrate with material such as cobalt-chromium material, that provide excellent bio-mechanical properties for stents without the need for adding relatively thick radiopaque surface layers. In stents, this advantage of having a thin surface layer can translate into less overall surface material and provide greater combined strength, flexibility, biocompatibility, and the potential for more complicated applications including vessel bifurcations, which benefit from wider openings between struts and flexibility about tortuous vessel branching paths. With reduced surface material exposed to body tissue and in the path of blood and other fluids, potential for restenosis or thrombosis is also reduced. The reduced material layer thickness promotes wider openings between struts 60, which can facilitate the insertion of stents within stents such as for a bifurcation procedure.

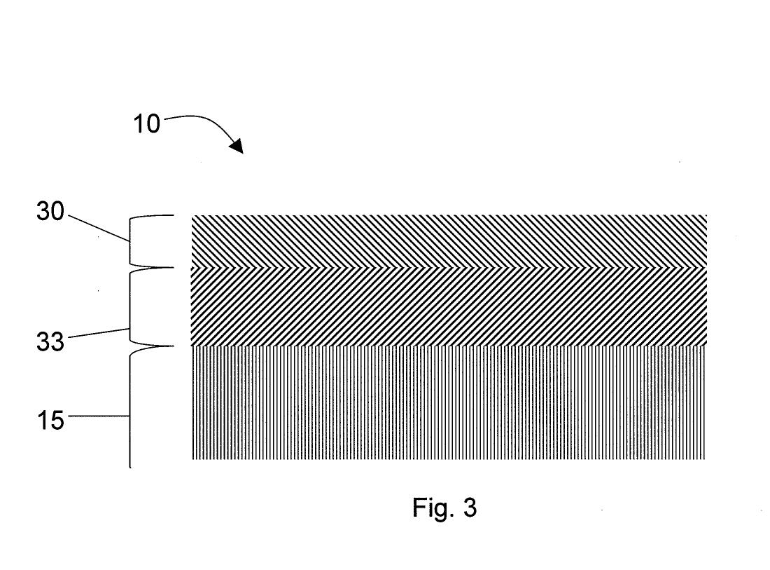

FIG. 3 is an illustrative cross-sectional view of a surface of an implantable device in accordance with another embodiment of the invention. While FIGS. 1 and 2B illustrate an adhesion layer 20 comprising both a base layer, or first portion 23, and a transition layer, or second portion 25, other applicable embodiments, such as the embodiment illustrated at FIG. 3, include a base layer or an adhesion layer 33, and no transition layer or second portion, disposed between the substrate 15 and capping layer 30. Referring to FIG. 3, an adhesion layer 33 is on the substrate 15, and a capping layer 30 is on the adhesion layer 33. In an embodiment, the adhesion layer 33 is directly on the substrate 15. In another embodiment, the capping layer 30 is directly on the adhesion layer 33. The adhesion layer 33 comprises an adhesion layer material, such as, for example, at least one of palladium and gold.

In an embodiment, the adhesion layer 33 consists essentially of adhesion layer materials to permit a strong bond to the substrate surface 15, such as palladium or gold, for example, 100% palladium or gold, or nearly 100% palladium or gold, or a mixture of palladium and gold, and comprises little or no capping layer material. In other embodiments, the adhesion layer 33 comprises a predominant proportion of adhesion layer material, for example, at least 50% palladium or gold.

In an embodiment, the adhesion layer 33 has a thickness of less than about 5000 angstroms. In another embodiment, the adhesion layer 33 has a thickness in the range of approximately 100 to 5000 angstroms, and preferably less than about 2500 angstroms, or otherwise sufficient to provide adequate bonding between the capping layer 30 and the substrate 15 while preserving the flexibility and formability of the stent. In another embodiment, the adhesion layer 33 has a thickness between about 500 and 2500 angstroms.

In an embodiment, a transition between the adhesion layer 33 and the substrate 15 has a thickness of about 10 atomic thicknesses or less. In another embodiment, the transition between the adhesion layer 33 and the substrate 15 has a thickness of about 5 atomic thicknesses or less. Preferably, the transition between the adhesion layer 33 and substrate 15 has a thickness of about 2 atomic thicknesses or less.

In an embodiment, a transition between the capping layer 30 and the adhesion layer 33 has a thickness of about 10 atomic thicknesses or less. In another embodiment, the transition between the capping layer 30 and the adhesion layer 33 has a thickness of about 5 atomic thicknesses or less. Preferably, the transition between the capping layer 30 and the adhesion layer 33 has a thickness of about 2 atomic thicknesses or less.

In an embodiment, the capping layer 30 comprises a predominant proportion of a capping layer material. In another embodiment, the capping layer 30 consists essentially of a capping layer material. In an embodiment, the capping layer material is a biocompatible material, for example, platinum. The capping layer 30, when comprised of a biocompatible material, can be in direct contact with human tissue.

In an embodiment, the adhesion layer 33 between the substrate 15 and the capping layer 30 comprises a predominant proportion of palladium throughout its thickness; that is, there is no gradated mixture of palladium and platinum. In another embodiment, the adhesion layer 33 between the substrate 15 and the capping layer 30 embodiment consists essentially of palladium.

In an embodiment, the adhesion layer 33 between the substrate 15 and the capping layer 30 comprises a predominant proportion of gold throughout its thickness from the substrate 15 to the capping layer 30, with no gradated mixture of gold and platinum. In another embodiment, the adhesion layer 33 between the substrate 15 and the capping layer 30 embodiment consists essentially of gold.

In an embodiment, an implantable device includes a substrate 15 comprising a predominant proportion of a radiopaque material, for example, cobalt-chromium material, an adhesion layer 33 comprising a predominant proportion of palladium, and a capping layer 30 comprising a predominant proportion of platinum.

In an embodiment, an implantable device includes a substrate 15 comprising a predominant proportion of a radiopaque material, for example, cobalt-chromium material, an adhesion layer 33 comprising a predominant proportion of gold, and a capping layer 30 comprising a predominant proportion of platinum.

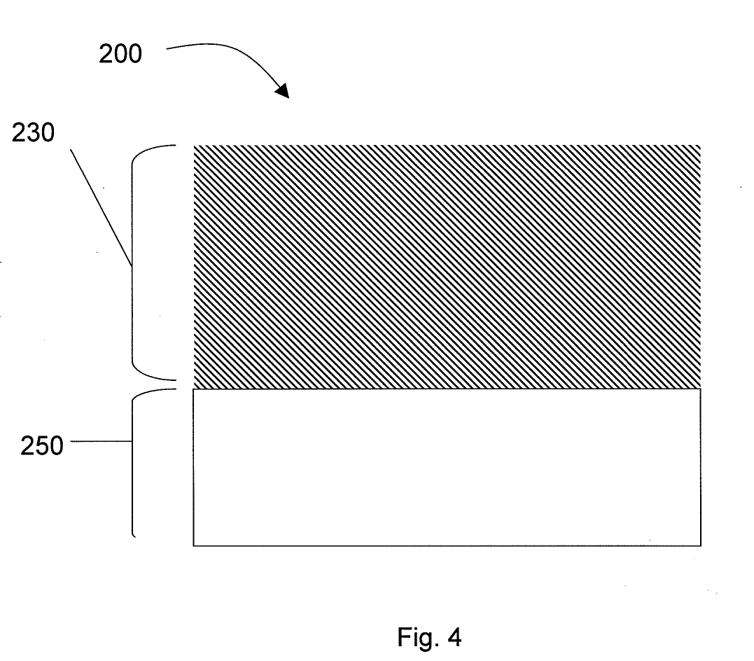

FIG. 4 is an illustrative cross-sectional view of a surface of an implantable device 200 in accordance with another embodiment of the invention. Referring to FIG. 4, an implantable device 200 comprises a substrate 250 and a biocompatable coating 230 that is directly on the substrate 250. In an embodiment, the biocompatible coating 230 comprises surface layers, such as the capping layer 30 and adhesion layers 20 or 33 disclosed in the embodiments described above in connection with FIGS. 1 and 3.

In an embodiment, the substrate 250 comprises cobalt-chromium material. The biocompatable coating 230, when formed directly on a substrate comprising cobalt-chromium material, has a thickness of less than 15,000 angstroms. In another embodiment, the biocompatable coating 230 has a thickness of less than about 10,000 angstroms. In another embodiment, the biocompatable coating 230 has a thickness of between about 2,500 and 5,000 angstroms. In another embodiment, the biocompatable coating 230 has a thickness of less than about 2500 angstroms. In another embodiment, the biocompatable coating 230 has a thickness of less than about 500 angstroms.

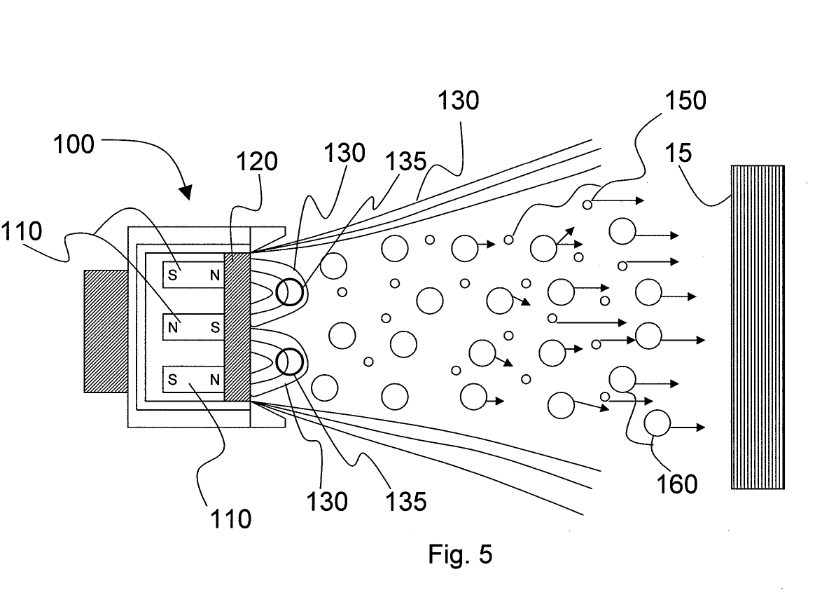

FIG. 5 is an illustrative view of surface layers being formed on a substrate 15 of an implantable device in accordance with an embodiment of the invention. Referring to FIG. 5, a magnetron 100 is used to apply the various aforementioned outer surface layers, including, for example, the adhesion layer 20 and capping layer 30 of FIG. 1, the adhesion layer 33 and capping layer 30 of FIG. 3, or the biocompatible coating 230 of FIG. 4, on the substrate 15. In an aspect of the invention, the magnetron 100 is an unbalanced magnetic field magnetron. The general methods of use and embodiments of magnetron systems in accordance with the invention are more fully described in U.S. Pat. No. 7,077,837, incorporated herein by reference in its entirety. The magnetron 100 includes a source 120 of atoms that is used to form at least one of the adhesion layer 20, 33 and the capping layer 30 on the substrate 15. The magnetron 100 creates an unbalanced magnetic field 130, wherein a plasma cloud 135 of metal atoms 160 and bombarding ions 150 is produced in the unbalanced magnetic field 130. The metal atoms 160 and bombarding ions 150 are supplied from a source 120 which is positioned in front of a plurality of magnets 110, which permits the magnetron 100 to create the unbalanced magnetic field 130. In this manner, the magnetron 100 can direct both the flux of metal atoms 160 and the flux of bombarding ions 150 onto the substrate 15 in a substantially collinear direction from the plasma cloud 135. As a result, the bombarding ions 150 impact and condense metal atoms 160, producing a substantially uniform layer of metal atoms on the substrate surface. Conventional balanced magnetic field magnetrons, on the other hand, generally depend on the use of independent sources for generating the coating metal atoms and bombarding ions, and can subsequently produce an inconsistent coating.

Furthermore, the methods disclosed in U.S. Pat. No. 7,077,837 can also improve the density of coatings relative to traditional IBAD (ion beam assisted deposition) which are limited to about a maximum density of between 92% to less than about 95% of full bulk density (wherein full bulk density is representative of a fully compacted non-porous material). In various embodiments of the invention, the unbalanced magnetrons can provide the above described coatings at about 95% to 98% of the full bulk density for the designated metal atoms. Classical IBAD applications (discrete non-colinear ion beam deposition) may employ fields of between about 0.8 keV to 1.5 keV. In embodiments of the invention, fields of between about 50 eV and 250 eV operating on ions supplied by a plasma cloud are directed to a target surface in substantially collinear fashion with the deposited metal atoms. Although such a field may provide less power per ion than do typical discrete ion beam methods, the reduced energy fields of various embodiments of the present invention are applied over a broader and more populated area (the plasma field) of ions and metal atoms, promoting greater uniformity in the thickness and density of the layers. The less energized ions are also less likely to cause back-sputtering (or loss of already deposited atoms on the surface coating) and can promote modest movement and shifting of the deposited metal atoms, thus providing enhanced density and uniformity of the layers.

In accordance with certain surface coating embodiments previously described, a magnetron 100 with unbalanced fields 130 can deposit metallic coating ions (e.g. palladium, gold, or platinum) onto a substrate surface (e.g. cobalt-chromium material) with the use of bombarding ions such as argon or xenon, such as, for example, for forming the first portion 23 of an adhesion layer 20 or capping layer 30 (shown in FIG. 1). In order to form mixed or gradated layers of multiple types of metals such as, for example, the second portion 25 of the adhesion layer 20, two or more magnetrons can be operated simultaneously to generate a flux of each of the respective metals.

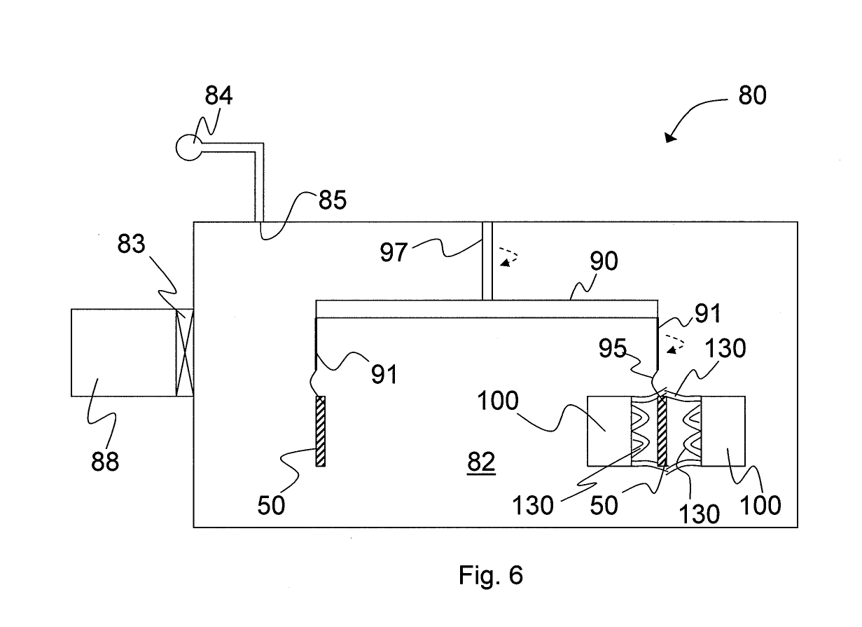

Referring to FIG. 6, an illustrative side-perspective schematic of an apparatus 80 for coating a substrate is shown according to an embodiment of the invention. Two or more magnetrons 100 are positioned relative to each other so that they can simultaneously direct a flux of different metal atom types toward the substrate of a stent 50. As a stent 50 is held in place between the fluxes 130 of magnetrons 100 by a fixture 91, which rotates stent 50 as the different metal atoms are deposited, thereby creating a substantially uniform coating of atoms mixed among the types deposited by each of the magnetrons 100. In an embodiment of the invention, a flexible attachment 95 allows stent 50 to vibrate in a substantially random manner, thus promoting further uniformity of the deposited layers. In an embodiment of the invention for creating a second portion or transition layer 25, one magnetron 100 of a two or more magnetron embodiment can deposit palladium or gold atoms while a second magnetron 100 can deposit platinum atoms. The magnetrons 100 can be controlled in synchronization (e.g. with the use of a processor/controller) to deposit desired ratios of each of the types of metals. For example, in an embodiment of the invention, a first magnetron can be controlled to gradually increase or decrease the concentration of a flux of first metal atoms, for example, palladium or gold, while a second magnetron generating a flux of second metal atoms, for example, platinum, can be controlled to gradually decrease or increase the concentration of the flux of metal atoms. For example, in forming the first portion 23 of the adhesion layer 20 shown in FIGS. 1 and 2A, 2B, the amount of first metal atoms being deposited can initially comprise 100% of the deposition on the substrate 15. To deposit a gradated mixture of first metal atoms and second metal atoms on the substrate 15, a mixture of first metal atoms and second metal atoms can be determined, by using the first magnetron to reduce the amount of first metal atoms being deposited on the substrate 15 while simultaneously using the second magnetron to increase the amount of second metal atoms being deposited on the substrate. The amount of second metal atoms being deposited can continue to increase, and the amount of first metal atoms can continue to decrease, until the second metal atoms comprise approximately 100% of the deposition, whereby a second portion 25 of the adhesion layer 20 is formed.

In various embodiments of the invention, one or more of the magnetrons 100 of the apparatus of FIG. 6 can be employed to apply the first portion 23 of the adhesion layer 20 of FIG. 1 or the adhesion layer 33 of FIG. 3, for example, comprising a predominant proportion of palladium or gold, and can be employed to apply the capping layer 30 comprising a predominant proportion of platinum. In an embodiment of the invention, two or more magnetrons 100 can provide a gradated, highly adhesive transition layer 25 that interfaces with the capping layer 30, for example of the type described above in connection with FIG. 1. In an embodiment, a capping layer 30 can then be formed on the adhesion layer 20 by using one or more magnetrons with the referenced methods to produce a layer such as with highly biocompatible materials (e.g. platinum). In an embodiment, a biocompatible coating 230 can be formed directly on the substrate 15, for example, of the type described above in connection with FIG. 4. Additional layers, including various biocompatible polymers, including drug-eluting polymers, may be applied over the metallic capping layer 30 or biocompatible coating 230.

Further referring to FIG. 6, an apparatus 80 is provided for processing multiple stents in a batch process using one or more magnetrons. Fixture 91 holding a stent 50 is attached at one end to a wheel 90 which is rotatable and driven via an axle 97 and an actuating mechanism (not shown). After one stent 50 has been coated by magnetrons 100, another stent 50 attached to wheel 90 can be actuated into place between magnetrons 100. In an embodiment of the invention, numerous stents 50 can be similarly attached to wheel 90 and coated in an automated manner with the aid of a programmed processor (not shown) that actuates wheel 90 and controls magnetrons 100, among various other components. Wheel 90 and attached stents 50 and magnetrons 100 are contained in a vacuum chamber 82. A vacuum of, for example, between 1E-3 to 1E-9 torr can be drawn from chamber 82 using a vacuum pump 88. Vacuum pumping may thereafter be throttled by a valve 83 and a noble gas, for instance, argon or xenon, may be introduced from a source 84 through a port 85 into chamber 82. The chamber 82 may continue to be filled with the noble gas to a pressure ranging from about 0.1 mtorr to about 100 mtorr. Next, an electrical charge of about −200 VDC to about −1000 VDC may be applied to stent 50 to rid its surface of oxides and other contaminants such as, for example, oxides that can develop on a cobalt-chromium or steel substrate during manufacture and affect the adhesiveness and safety of the device. This pre-cleaning process of the device may last from about 5 to about 60 minutes, depending on the initial cleanliness of a stent 50. Once the ion pre-cleaning process is completed, the coating process using multiple magnetrons 100 may begin such as in accordance with the details discussed above and in connection with U.S. Pat. No. 7,077,837 incorporated by reference above. In an embodiment of the invention, the techniques illustrated above can be used for the purposes of adding additional layers of metals, polymers, and/or therapeutic agents in addition to the surface layers disclosed herein. The surface layers disclosed herein can provide reduced thicknesses and improved adhesion, uniformity, and purity of preferred metals so as to improve the adhesion of the additional layers and the overall biocompatibility and safety of an implantable device.

It will be understood by those with knowledge in related fields that uses of alternate or varied materials and modifications to the methods disclosed are apparent. This disclosure, including the claims herein, are intended to cover these and other variations, uses, or other departures from the specific embodiments as come within the art to which the invention pertains.

1. An implantable device comprising:
a substrate;
an adhesion layer comprising a portion with a predominant proportion of palladium, the portion of the adhesion layer with a predominant proportion of palladium directly on the substrate; and
a capping layer comprising a capping layer material, the capping layer on the adhesion layer.

2. The device of claim 1, wherein the capping layer material comprises a biocompatible material.

3. The device of claim 2, wherein the biocompatible material comprises at least one of platinum, platinum-iridium, tantalum, titanium, and alloys thereof.

4. The device of claim 2, wherein the biocompatible material comprises at least one of tin, indium, palladium, gold and alloys thereof.

5. The device of claim 1, wherein the capping layer material comprises a predominant proportion of platinum.

6. The device of claim 1, wherein the adhesion layer between the substrate and the capping layer has a thickness of less than about 5000 angstroms.

7. The device of claim 1, wherein at least one of the capping layer and the adhesion layer has a thickness between about 100 and 5000 angstroms.

8. The device of claim 7, wherein at least one of the capping layer and the adhesion layer has a thickness between about 500 and 2500 angstroms.

9. The device of claim 1, wherein the capping layer has a thickness of less than about 2500 angstroms.

10. The device of claim 1, wherein at least one of the adhesion layer and the capping layer is substantially of a density greater than about 95% full bulk density.

11. The device of claim 1, wherein at least one of the adhesion layer and the capping layer is substantially of a density equal to or greater than about 97% full bulk density.

12. The device of claim 1, wherein the substrate comprises a highly radiopaque material.

13. The device of claim 12, wherein the highly radiopaque material comprises cobalt-chromium material.

14. The device of claim 1, wherein the substrate comprises a metallic material including at least one of stainless steel, nickel-based steel, cobalt-chromium, titanium, nitinol, and alloys thereof.

15. The device of claim 1, wherein the adhesion layer comprises a first portion that is directly on the substrate and a second portion that is directly on the first portion, and wherein the second portion is between the first portion and the capping layer.

16. The device of claim 15, wherein the second portion comprises a gradated mixture of palladium and capping layer material, wherein the gradated mixture of palladium and capping layer material includes a high concentration of palladium and a low concentration of capping layer material in a region proximal to the first portion of the adhesion layer, and wherein the gradated mixture of palladium and capping layer material includes a low concentration of palladium and a high concentration of capping layer material in a region proximal to the capping layer.

17. The device of claim 1, wherein the capping layer is directly on the adhesion layer.

18. The device of claim 1, wherein the adhesion layer comprises a predominant proportion of palladium throughout its thickness.

18. The device of claim 1, wherein the capping layer material comprises a material other than palladium.

20. The device of claim 1 further comprising a polymer layer on the capping layer.

21. The device of claim 1, wherein the implantable device comprises a flexible body.

22. The device of claim 1, wherein the implantable device is an intravascular stent.

23. An implantable device comprising:
a substrate;
an adhesion layer comprising a portion with a predominant proportion of gold, the portion of the adhesion layer with a predominant proportion of gold directly on the substrate; and
a capping layer comprising a capping layer material, the capping layer on the adhesion layer, wherein the adhesion layer between the substrate and the capping layer has a thickness of less than about 5000 angstroms.

24. The device of claim 23, wherein the capping layer material comprises a biocompatible material.

25. The device of claim 24, wherein the biocompatible material comprises at least one of platinum, platinum-iridium, tantalum, titanium, and alloys thereof.

26. The device of claim 24, wherein the biocompatible material comprises at least one of tin, indium, palladium, gold and alloys thereof.

27. The device of claim 23, wherein the capping layer material comprises a predominant proportion of platinum.

28. The device of claim 23, wherein at least one of the capping layer and the adhesion layer has a thickness between about 100 and 5000 angstroms.

29. The device of claim 28, wherein at least one of the capping layer and the adhesion layer has a thickness between about 500 and 2500 angstroms.

30. The device of claim 23, wherein the capping layer has a thickness of less than about 2500 angstroms.

31. The device of claim 23, wherein at least one of the adhesion layer and the capping layer is substantially of a density greater than about 95% full bulk density.

32. The device of claim 23, wherein at least one of the adhesion layer and the capping layer is substantially of a density equal to or greater than about 97% full bulk density.

33. The device of claim 23, wherein the substrate comprises a highly radiopaque material.

34. The device of claim 33, wherein the highly radiopaque material includes cobalt-chromium material.

35. The device of claim 23, wherein the substrate comprises a metallic material including at least one of stainless steel, nickel-based steel, cobalt-chromium, titanium alloys, nitinol, and alloys thereof.

36. The device of claim 23, wherein the adhesion layer comprises a first portion that is directly on the substrate and a second portion that is directly on the first portion, and the second portion is between the first portion and the capping layer.

37. The device of claim 36, wherein the second portion comprises a gradated mixture of gold and capping layer material, wherein the gradated mixture of gold and capping layer material includes a high concentration of gold and a low concentration of capping layer material in a region proximal to the first portion of the adhesion layer, and wherein the gradated mixture of gold and capping layer material includes a low concentration of gold and a high concentration of capping layer material in a region proximal to the capping layer.

38. The device of claim 23, wherein the capping layer is directly on the adhesion layer.

39. The device of claim 23, wherein the adhesion layer comprises a predominant proportion of gold throughout its thickness.

40. A method of providing a surface on an implantable device comprising:
providing a substrate of the implantable device;
providing an adhesion layer comprising a portion with a predominant proportion of palladium directly on the substrate by simultaneously directing a flux of palladium atoms and a flux of bombarding ions toward the substrate; and
providing a capping layer comprising a capping layer material on the adhesion layer by directing a flux of capping layer material atoms and a flux of bombarding ions toward the provided adhesion layer.

41. The method of claim 40 wherein the bombarding ions are directed in substantially collinear fashion toward the substrate with respect to said fluxes of palladium or capping material atoms:

42. The method of claim 40 wherein providing the adhesion layer comprises:
providing a first portion of the adhesion layer directly on the substrate, the first portion of the adhesion layer comprising the predominant proportion of palladium; and
providing a second portion of the adhesion layer directly on the first portion, the second portion comprising a gradated mixture of palladium and capping layer material between the first portion and the capping layer.

43. The method of claim 42, wherein the gradated mixture includes a high concentration of palladium and a low concentration of capping layer material in a region proximal to the first portion of the adhesion layer by providing a greater proportion of palladium atoms than capping layer material atoms, and wherein the gradated mixture includes a low concentration of palladium and a high concentration of capping layer material in a region proximal to the capping layer by providing a greater proportion of capping layer material atoms than palladium atoms.

44. The method of claim 42, wherein the gradated mixture is provided by simultaneously directing the fluxes of palladium atoms, capping layer material atoms, and bombarding ions toward the substrate.

45. The method of claim 40, wherein forming the adhesion layer comprises using at least one magnetron to direct the fluxes of palladium atoms and the capping layer material atoms.

46. The method of claim 45, wherein the at least one magnetron comprises an unbalanced magnetron.

47. The method of claim 40, wherein the capping layer is substantially biocompatible.

48. The method of claim 40, wherein the capping layer material atoms are platinum atoms.

49. The method of claim 40, wherein the adhesion layer between the substrate and the capping layer has a thickness of less than about 5000 angstroms.

50. The method of claim 40, wherein at least one of the capping layer and the adhesion layer has a thickness between about 100 and 5000 angstroms.

51. The method of claim 40, wherein at least one of the capping layer and the adhesion layer has a thickness of less than about 2500 angstroms.

52. The method of claim 40, wherein providing the capping layer comprises forming the capping layer directly on the adhesion layer.

53. The method of claim 40, wherein providing the adhesion layer comprises providing the adhesion layer to comprise a predominant proportion of palladium throughout its thickness.

54. The method of claim 40, wherein the adhesion layer is substantially of a density greater than about 95% full bulk density.

55. The method of claim 40, wherein the capping layer is substantially of a density greater than about 95% full bulk density.

56. The method of claim 40, wherein the adhesion layer is substantially of a density equal to or greater than about 97% full bulk density.

57. The method of claim 40, wherein the capping layer is substantially of a density equal to or greater than about 97% full bulk density.

58. A method of providing a surface on an implantable device comprising:
providing a substrate of the implantable device;
providing an adhesion layer comprising a portion having a predominant proportion of gold directly on the substrate by simultaneously directing a flux of gold atoms and a flux of bombarding ions toward the substrate; and
providing a capping layer comprising a capping layer material on the adhesion layer by directing a flux of capping layer material atoms and a flux of bombarding ions toward the provided adhesion layer, the adhesion layer between the substrate and the capping layer having a thickness of less than about 5000 angstroms.

59. The method of claim 58 wherein the bombarding ions are directed in substantially collinear fashion toward the substrate with respect to said fluxes of gold atoms or capping material atoms.

60. The method of claim 58 wherein providing the adhesion layer comprises:
providing a first portion of the adhesion layer directly on the substrate, the first portion of the adhesion layer comprising the predominant proportion of gold; and
providing a second portion of the adhesion layer directly on the first portion, the second portion comprising a gradated mixture of gold and capping layer material between the first portion and the capping layer.

61. The method of claim 58, wherein the capping layer is substantially biocompatible.

62. The method of claim 58, wherein the capping layer material atoms are platinum atoms.

63. The method of claim 58, wherein at least one of the capping layer and the adhesion layer has a thickness between about 100 and 5000 angstroms.

64. The method of claim 58, wherein at least one of the capping layer and the adhesion layer has a thickness of less than about 2500 angstroms.

65. The method of claim 58, wherein providing the capping layer comprises forming the capping layer directly on the adhesion layer.

66. The method of claim 58, wherein at least one of the capping layer or adhesion layer is substantially of a density greater than about 95% full bulk density.

67. The method of claim 58, wherein at least one of the capping layer or adhesion layer is substantially of a density greater than or equal to about 97% full bulk density.

68. The device of claim 1, wherein the capping layer material consists essentially of platinum.

69. An implantable device comprising:
a substrate; and
a coating directly on the substrate, the coating comprising a capping layer of essentially platinum, wherein the coating has a thickness of less than about 15,000 angstroms.

70. The implantable device of claim 69 wherein the coating has a thickness of between about 100 and 5000 angstroms.

71. The implantable device of claim 69 wherein the coating comprises an adhesion layer with a predominant proportion of palladium, the capping layer of essentially platinum directly on the adhesion layer.

72. A method of providing a surface on an implantable device comprising:
providing a substrate; and
forming a coating directly on the substrate, the coating comprising a capping layer of essentially platinum, wherein the coating has a thickness of less than about 15,000 angstroms.
